(19) United States
(12) Patent Application Publication
(10) Pub. No.: US 2017/0204449 A1
(43) Pub. Date: Jul. 20, 2017

(54) METHOD FOR CHARACTERIZING BACTERIAL MUTANTS

(71) Applicant: DISCUVA LIMITED, Cambridge (GB)

(72) Inventors: David Hugh Williams, Melbourn (GB); John Richard Wain, Melbourn (GB); Stuart Robert Woods, Longstanton (GB)

(73) Assignee: DISCUVA LIMITED, Cambridge (GB)

(21) Appl. No.: 15/415,361

(22) Filed: Jan. 25, 2017

Related U.S. Application Data
(63) Continuation of application No. PCT/GB2015/052079, filed on Jul. 17, 2015.

(30) Foreign Application Priority Data
Jul. 25, 2014 (GB) 1413198.1

(51) Int. Cl. C12Q 1/18; C12N 15/10; C12Q 1/68

(52) U.S. Cl. CPC C12Q 1/18; C12Q 1/689; C12N 15/1082; C12Q 2600/158; G01N 2333/195; G01N 2500/10

(57) ABSTRACT

Disclosed is a method for characterizing the effect of an antibiotic on a bacterium, the method comprising the steps of: (a) generating a pool of mutant bacteria by transposon mutagenesis of a culture of said bacterium with an activating transposon (TnA) which comprises an outward-facing promoter (TnAP) capable of increasing transcription of a gene at or near its insertion site in the DNA of said bacterium; (b) growing bacteria from the mutant pool in the presence of different amounts of said antibiotic to produce two or more test cultures; (c) sequencing mRNA transcripts produced by TnAP in each of said test cultures to produce an mRNA transcript profile for each of the test cultures; and (d) comparing the mRNA transcript profiles of the test cultures.

### RELATED APPLICATIONS

This application is a continuation of, and claims the benefit of priority to, international application PCT/GB2015/052079, filed Jul. 17, 2015, which was published under PCT Article 21(2) in English, and which claims priority to United Kingdom application 1413198.1, filed Jul. 25, 2014, the entire contents of each of which are incorporated by reference herein.

### FIELD OF THE INVENTION

The present invention relates to methods for characterizing the effect of an antibiotic on a bacterium and for identifying antibiotic targets in bacteria, to methods for identifying antibiotics and to processes for producing antibiotics and pharmaceutical compositions comprising said antibiotics.

### BACKGROUND TO THE INVENTION

There is an urgent need for new antibiotics to counter the emergence of new pathogens and resistance to existing antimicrobial drugs. The identification of the targets of candidate antibiotics is critical, since such information can provide access to a large number of functionally related novel drug families. For example, the discovery of the penicillin-binding proteins as targets of penicillin led to the development of a large family of antibiotics, including multiple generations of cephalosporins, penicillins and carbapenems (see Schmid (2006) Nature Biotechnology 24(4): 419-420).

Transposon directed insertion-site sequencing (TraDIS—see Langridge et al. (2009) Genome Research 19: 2308-2316) has recently been described and used to identify: (a) essential genes; (b) genes advantageous (but not essential) for growth; (c) genes disadvantageous for growth under particular conditions; and (d) genes involved in conferring tolerance to certain conditions (“niche-specific” essential genes). Similar techniques have been described in e.g. Gawronski et al. (2009) PNAS 106: 16422-16427; Goodman et al. (2009) Cell Host Microbe 6: 279-289; van Opijnen et al. (2009) Nat. Methods 6: 767-772 and Gallagher et al. (2011) mBio 2(1):e00315-10, and such techniques are now collectively dubbed “Tn-seq” methods.

However, an important class of antibiotic targets are gene products involved in cellular processes essential for viability in the growth conditions used. Such targets cannot be identified by Tn-seq (including TraDIS), since transposon insertions into essential genes (including those serving as antibiotic targets) are not significantly represented in the initial mutant pool. Thus, differences in transposon distribution after growth of the mutant pool with or without (or with varying amounts of) antibiotic would not arise, with the result that Tn-seq cannot distinguish between an essential gene and an essential gene serving as an antibiotic target.

There is therefore a need for high-throughput functional screens for antibiotic targets which are capable of identifying essential genes serving as antibiotic targets.

WO2012/150432 describes a method for identifying an essential gene which serves as an antibiotic target in a bacterium comprising the steps of:
(a) generating a pool of mutant bacteria by transposon mutagenesis with an activating transposon (Tn_{A}), wherein the Tn_{A} comprises a promoter such that transposon insertion into bacterial DNA increases the transcription of a gene at or near the insertion site;
(b) growing bacteria from the mutant pool in the presence of different amounts of said antibiotic to produce two or more test cultures; and
(c) comparing the distribution of Tn_{A} insertions between test cultures to identify a putative essential gene serving as a target of said antibiotic in said bacterium.

It has now been discovered that the quality of the data obtained via such methods is greatly improved and enriched by characterizing the Tn_{A} insertions at the RNA level. This permits the collection of data which directly reports the effect of Tn_{A} insertion on gene expression, while also providing a valuable insight into the effects of Tn_{A} insertion into the non-coding strand of the bacterial DNA—in particular, it permits an analysis of the effects of antisense transcripts (e.g. down-regulation) on gene expression.

### SUMMARY OF THE INVENTION

A method for characterizing the effect of an antibiotic on a bacterium, the method comprising the steps of:
(a) generating a pool of mutant bacteria by transposon mutagenesis of a culture of said bacterium with an activating transposon (Tn_{A}) which comprises an outward-facing promoter (Tn_{A}P) capable of increasing transcription of a gene at or near its insertion site in the DNA of said bacterium;
(b) growing bacteria from the mutant pool in the presence of different amounts of said antibiotic to produce two or more test cultures;
(c) sequencing mRNA transcripts produced by Tn_{A}P in each of said test cultures to produce an mRNA transcript profile for each of the test cultures; and
(d) comparing the mRNA transcript profiles of the test cultures.

The mRNA transcript profile may comprise a determination of the sequence of one or more antisense transcripts arising from TnA insertion into an insertion site within a noncoding, anti-sense strand of the DNA of said bacterium. Such antisense transcripts may suppress gene expression, for example they may suppress expression of genes by binding to complementary mRNA encoded by the corresponding coding (sense) strand of the DNA of said bacterium.

In another aspect, there is provided a method of identifying an antibiotic comprising identifying an essential gene which serves as a target of said antibiotic according to a method of the invention.

In a further aspect, there is provided a process for producing an antibiotic comprising identifying an antibiotic by a method comprising identifying an essential gene which serves as a target of said antibiotic according to a method of the invention. Such a process may optionally further comprise the step of synthesising said antibiotic, and may optionally further comprise mixing the synthesised antibiotic with a pharmaceutically acceptable excipient to produce a pharmaceutical composition.

The use of an activating transposon ensures that transposon insertions into essential genes are represented in the initial mutant pool, since transposon insertion can now result in gene activation rather than insertional inactivation. Thus, the effect of the presence of antibiotic during subsequent culture of the mutant pool on transposon distribution and gene expression can be studied (and the identity of the gene target(s) thereby determined).

Other aspects and preferred embodiments of the invention are defined and described in the other claims set out below.

### BRIEF DESCRIPTION OF THE DRAWINGS

FIG. 1 shows a genetic map of plasmid pAMICS1-Cm-PrrnB.

FIG. 2 shows results from an analysis of transcription of genomic regions.

FIG. 3 shows results from an analysis of transcription.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

The term gene is a term describing a hereditary unit consisting of a sequence of DNA that occupies a specific location on a chromosome or plasmid and determines a particular characteristic in an organism. A gene may determine a characteristic of an organism by specifying a polypeptide chain that forms a protein or part of a protein (structural gene); or encode an RNA molecule; or regulate the operation of other genes or repress such operation; or affect phenotype by some other as yet undefined mechanism.

The term genomic DNA is a term of art used herein to define chromosomal DNA as distinct from extra-chromosomally-maintained (e.g. plasmid) DNA.

The term genome is a term of art used herein to define the entire genetic complement of an organism, and so includes chromosomal, plasmid, prophage and any other DNA.

The term Gram-positive bacterium is a term of art defining a particular class of bacteria that are grouped together on the basis of certain cell wall staining characteristics and subclasses are defined by the DNA base composition, low G+C Gram-positive bacterium

subclass includes Streptococcus spp., Staphylococcus spp., Listeria spp., Bacillus spp., Clostridium spp., Enterococcus spp. and Lactobacillus spp.). The high G+C Gram-positive bacterium subclass includes actinomycetes (actinobacteria) including Actinomyces spp., Arthrobacter spp., Corynebacterium spp., Frankia spp., Micrococcus spp., Micromonospora spp., Mycobacterium spp., Nocardia spp., Propionibacterium spp. and Streptomyces spp.

The term Gram-negative bacterium is a term of art defining a particular class of bacteria that are grouped together on the basis of certain cell wall staining characteristics. Examples of Gram-negative bacterial genera include Klebsiella, Acinetobacter, Escherichia, Pseudomonas, Enterobacter and Neisseria.

As used herein, the term “essential gene” is a term of art defining a particular class of genes the products of which are necessary for viability, either under all conditions or under the conditions of growth used. An important subclass of essential gene are those encoding products (e.g. proteins, peptides and regulatory polynucleotides) which contribute to metabolic processes essential for viability under important growth conditions (for example, and in the case of pathogenic bacteria, under conditions which prevail during infection or multiplication in the host).

### Antibiotics and Antibiotic Targets

The antibiotic used to produce the test cultures of the invention is typically a novel investigational antibiotic (anti-bacterial chemotherapeutic agent), the mechanism of action (and hence biological target(s)) of which are unknown. In many applications, the antibiotic is selected from combinatorial libraries, natural product libraries, defined chemical entities, peptides, peptide mimetics and oligonucleotides.

The antibiotic target identified according to the invention is an essential gene/gene product, and may therefore be involved in one or more of the following biological processes in the bacterial host:
(a) cell division;
(b) DNA replication (including polymerization and supercoiling);
(c) transcription (including priming, elongation and termination);
(d) translation (including ribosome components, initiation, elongation and release);
(e) biosynthetic pathways (including peptidoglycan and fatty acids);
(f) plasmid addiction also known as toxin/antitoxin;
(g) cell wall assembly; and/or
(h) bacterial cell integrity.

### Bacteria for Use in the Methods of the Invention

The methods of the invention may be applied to identify an antibiotic target in any bacterium. Thus, the methods of the invention find application in the identification of antibiotic targets in: (a) Gram-positive, Gram-negative and/or Gram-indeterminate (Gram-variable) bacteria; (b) spore-forming bacteria; (c) non-spore forming bacteria; (d) filamentous bacteria; (e) intracellular bacteria; (f) obligate aerobes; (g) obligate anaerobes; (h) facultative anaerobes; (i) microaerophilic bacteria and/or (f) opportunistic bacterial pathogens.

In certain embodiments, the methods of the invention are applied to identify an antibiotic target in bacteria of the following genera: Acinetobacter (e.g. A. baumannii); Aeromonas (e.g. A. hydrophila); Bacillus (e.g. B. anthracis); Bacteroides (e.g. B. fragilis); Bordetella (e.g. B. pertussis); Borrelia (e.g. B. burgdorferi); Brucella (e.g. B. abortus, B. canis, B. melitensis and B. suis); Burkholderia (e.g. B. cepacia complex); Campylobacter (e.g. C. jejuni); Chlamydia (e.g. C. trachomatis, C. suis and C. muridarum); Chlamydophila (e.g. (e.g. C. pneumoniae, C. pecorum, C. psittaci, C. abortus, C. felis and C. caviae); Citrobacter (e.g. C. freundii); Clostridium (e.g. C. botulinum, C. difficile, C. perfringens and C. tetani); Corynebacterium (e.g. C. diphteriae and C. glutamicum); Enterobacter (e.g. E. cloacae and E. aerogenes); Enterococcus (e.g. E. faecalis and E. faecium); Escherichia (e.g. E. coli); Flavobacterium; Francisella (e.g. F. tularensis); Fusobacterium (e.g. F. necrophorum); Haemophilus (e.g. H. somnus, H. influenzae and H. parainfluenzae); Helicobacter (e.g. H. pylori); Klebsiella (e.g. K. oxytoca and K. pneumoniae), Legionella (e.g. L. pneumophila); Leptospira (e.g. L. interrogans); Listeria (e.g. L. monocytogenes); Moraxella (e.g. M. catarrhalis); Morganella (e.g. M. morganii); Mycobacterium (e.g. M. leprae and M. tuberculosis); Mycoplasma (e.g. M. pneumoniae); Neisseria (e.g. N. gonorrhoeae and N. meningitidis); Pasteurella (e.g. P. multocida); Peptostreptococcus; Prevotella; Proteus (e.g. P. mirabilis and P. vulgaris), Pseudomonas (e.g. P. aeruginosa); Rickettsia (e.g. R. rickettsii); Salmonella (e.g. serotypes. Typhi and Typhimurium); Serratia (e.g. S. marcesens); Shigella (e.g. S. flexnaria, S. dysenteriae and S. sonnei); Staphylococcus (e.g. S. aureus, S. haemolyticus, S. intermedius, S. epidermidis and S. saprophyticus); Stenotrophomonas (e.g. S. maltophila); Streptococcus (e.g. S. agalactiae, S. mutans, S. pneumoniae and S. pyogenes); Treponema (e.g. T. pallidum); Vibrio (e.g. V. cholerae) and Yersinia (e.g. E pestis).

The methods of the invention may be used to identify an antibiotic target in multi-drug resistant bacteria, including, but not limited to penicillin-resistant, methicillin-resistant, quinolone-resistant, macrolide-resistant, and/or vancomycin-resistant bacterial strains, including for example penicillin-, methicillin-, macrolide-, vancomycin-, and/or quinolone-resistant Streptococcus pneumoniae; penicillin-, methicillin-, macrolide-, vancomycin-, and/or quinolone-resistant Staphylococcus aureus; penicillin-, methicillin-, macrolide-, vancomycin-, and/or quinolone-resistant Streptococcus pyogenes; and penicillin-, methicillin-, macrolide-, vancomycin-, and/or quinolone-resistant enterococci.

Thus, methods of the invention may be used to identify an antibiotic target in methicillin-resistant Staphylococcus aureus (MRSA), for example selected from any of C-MSRA1, C-MRSA2, C-MRSA3, C-MSRA4, Belgian MRSA, Swiss MRSA and any of the EMRSA strains.

The compounds of the invention may be used to identify an antibiotic target in both high G+C Gram-positive bacteria and in low G+C Gram-positive bacteria.

The methods of the invention find particular application in the identification of an antibiotic target in a bacterium selected from Klebsiella pneumoniae, Acinetobacter baumanii, Escherichia coli (including ST131), Enterococcus faecalis, Enterococcus faecium, Pseudomonas aeruginosa, Enterobacter cloacae, Enterobacter aerogenes and Neisseria gonorrhoeae.

Particularly preferred are methods of identifying an antibiotic target in Klebsiella pneumoniae, Acinetobacter baumanii or Escherichia coli.

### Mutant Pools

The methods of the invention involve generating a pool of mutant bacteria by transposon mutagenesis. The size of the mutant pool affects the resolution of the method: as the pool size increases, more and more different genes with Tn_{A} insertions will be represented (and so effectively assayed). As the pool size decreases, the resolution of the method reduces, genes will be less effectively assayed, and more and more genes will not be assayed at all.

Ideally, the mutant pool generated in the methods of the invention is comprehensive, in the sense that insertions into every gene are represented. The number of Tn_{A} insertion mutants (i.e. the mutant pool size) required to achieve this depends on various factors, including: (a) the size of the bacterial genome; (b) the average size of the genes; and (c) any Tn_{A} insertion site bias.

With regard to the latter, some areas of bacterial genomes attract a low frequency of insertion (especially GC-rich regions). Thus, insertion frequencies and pool sizes large enough to ensure that insertions into insertion-refractory regions are preferred.

In general, a minimum insertion rate of one transposon per 25 bp is required to achieve a comprehensive pool/library, which typically entails a minimum pool size for bacteria having a genome size of 4 to 7 Mb of 0.5×10⁵ to 1×10⁵, for example 5×10⁵, preferably at least about 1×10⁶ mutants. In many cases, 1×10⁶ mutants will allow identification of ˜300,000 different insertion sites and correspond to 1 transposon insertion every 13 to 23 bp (or about 40-70 different insertion sites per gene).

However, the methods of the invention do not necessarily require a comprehensive mutant pool (in the sense defined above) in order to return useful information as to the identity of antibiotic drug targets. Rather, pool sizes less than the ideal comprehensive pool may be used, provided that a reduction in resolution (and attendant failure to assay certain genes) can be tolerated. This may be the case, for example, where the method is designed to be run iteratively until the target is identified: in such embodiments the effective pool size grows with each iteration of the method.

### Transposon Mutagenesis

Transposons, sometimes called transposable elements, are polynucleotides capable of inserting copies of themselves into other polynucleotides. The term transposon is well known to those skilled in the art and includes classes of transposons that can be distinguished on the basis of sequence organisation, for example short inverted repeats at each end; directly repeated long terminal repeats (LTRs) at the ends; and polyA at 3′ ends of RNA transcripts with 5′ ends often truncated.

Transposomes are transposase-transposon complexes wherein the transposon does not encode transposase. Thus, once inserted the transposon is stable. Preferably, in order to ensure mutant pool stability, the transposon does not encode transposase and is provided in the form of a transposome (i.e. as a complex with transposase enzyme), as described below.

As used herein, the term “activating transposon” (hereinafter abbreviated “Tn_{A}”) defines a transposon which comprises a promoter such that transposon insertion increases the transcription of a gene at or near the insertion site. Examples of such transposons are described in Troeschel et al. (2010) Methods Mol Biol. 668:117-39 and Kim et al. (2008) Curr Microbiol. 57(4): 391-394.

The activating transposon/transposome can be introduced into the bacterial genome (including chromosomal and/or plasmid DNA) by any of a wide variety of standard procedures which are well-known to those skilled in the art. For example, Tn_{A} transposomes can be introduced by electroporation (or any other suitable transformation method).

Preferably, the transformation method generates 1×10³ to 5×10³ transformants/ng DNA, and such transformation efficiencies are generally achievable using electroporation.

Alternatively, transposon mutagenesis using Tn_{A} may be performed in vitro and recombinant molecules transformed/transfected into bacterial cells. In such embodiments, transposomes can be prepared according to a standard protocol by mixing commercially available transposase enzyme with the transposon DNA fragment. The resulting transposomes are then mixed with extra-chromosomal DNA of interest to allow transposition, then the DNA is introduced into a host bacterial strain using electrotransformation to generate a pool of extra-chromosomal DNA transposon mutants.

In embodiments where mutagenesis is performed in vitro, it is possible to mix transposomes with genomic DNA in vitro and then introduce the mutagenized DNA (optionally, after fragmentation and/or circularization) into the host bacterial strain (e.g. by electroporation) whereupon endogenous recombination machinery incorporates it into the genome. Such an approach may be particularly useful in the case of bacteria which are naturally competent (e.g. Acinetobacter spp.) and/or can incorporate DNA via homologous crossover (e.g. double crossover) recombination events.

### Activating Transposons for Use in the Methods of the Invention

Any suitable activating transposon may be used in the methods of the invention. Suitable transposons include those based on Tn3 and the Tn3-like (Class II) transposons including γδ (Tn1000), Tn501, Tn2501, Tn21, Tn917 and their relatives. Also Tn10, Tn5, TnphoA, Tn903, bacteriophage Mu and related transposable bacteriophages. A variety of suitable transposons are also available commercially, including for example the EZ-Tn5™<R6Kγori/KAN-2> transposon.

Preferred transposons are those which carry antibiotic resistance genes (which may be useful in identifying mutants which carry a transposon) including Tn5, Tn10 and TnphoA. For example, Tn10 carries a tetracycline resistance gene between its IS elements while Tn5 carries genes encoding polypeptides conferring resistance to kanamycin, streptomycin and bleomycin. Other suitable resistance genes include those including chloramphenicol acetyltransferase (conferring resistance to chloramphenicol).

It is of course possible to generate new transposons by inserting different combinations of antibiotic resistance genes between IS elements, or by inserting combinations of antibiotic resistance genes between transposon mosaic ends (preferred), or by altering the polynucleotide sequence of the transposon, for example by making a redundant base substitution or any other type of base substitution that does not affect the transposition or the antibiotic resistance characteristics of the transposon, in the coding region of an antibiotic resistance gene or elsewhere in the transposon. Such transposons are included within the scope of the invention.

In many embodiments, a single transposon is used to generate the mutant pool. However, as explained above, the number of Tn insertion mutants (i.e. the mutant pool size) required to achieve a comprehensive pool or library depends inter alia on any Tn insertion site bias. Thus, in cases where the transposon insertion site bias occurs, two or more different transposons may be used in order to reduce or eliminate insertion site bias. For example, a combination of two different transposons based on Tn5 and Tn10 may be employed.

### Promoters for Use in Activating Transposons

The nature of the promotor present in the Tn_{A} is dependent on the nature of the transposon and the ultimate bacterial host. Generally, an efficient, outward-oriented promoter which drives high level transcription of DNA near or adjacent to the insertion site is chosen.

The promoter may include: (a) a Pribnow box (−10 element); (b) a −35 element and/or (c) an UP element.

For example, the lac promoter can be used with the EZ-Tn5™<R6Kγori/KAN-2> transposon, and such constructs are suitable for assay of e.g. Escherichia coli, Enterobacter spp. and other members of the family Enterobacteriaceae such as Klebsiella spp. Other suitable promoters include: rplJ (large ribosomal subunit protein; moderate strength promoter); tac (artificial lac/trp hybrid; strong promoter) and rrnB (ribosomal RNA gene promoter; very strong promoter).

### Use of MultipleTn_{A}/Multiple Promoters

In some embodiments of the invention, the bacterial genome is probed with a mixture of different activating transposons which have outward facing promoters of different strengths. In some circumstances, a broader range of genes involved in antibiotic resistance and/or sensitivity are recovered if a mixture of activating transposons with at least three different promoters of progressively decreasing strength are employed to generate the mutant pool. In such circumstances, the use of a plurality of activating transposons with promoters of varying strength ensures that transposon insertions occur substantially in all genes involved in mediating antibiotic sensitivity or resistance are represented in the initial mutant pool, since transposon insertion can now result in gene activation to yield an appropriate level of transcription (neither too high, nor too low). Thus, the effect of the presence of antibiotic during subsequent culture of the mutant pool on transposon distribution can be studied (and the identity of the relevant gene target(s) thereby determined).

In such embodiments, a wide variety of promoters may be used provided that at least three different promoters are used wherein the relative strength of said promoters is: Tn_{A}P1>Tn_{A}P2>Tn_{A}P3; such that transposon insertion into bacterial DNA generates a pool of mutant bacteria in which one or more genes are transcribed from Tn_{A}P1, one or more genes are transcribed from Tn_{A}P2 and one or more genes are transcribed from Tn_{A}P3.

Preferably, Tn_{A}P1 is a strong promoter, Tn_{A}P2 a medium-strength promoter and Tn_{A}P3 a weak promoter in the mutagenized bacteria under the conditions used for growth of the mutant pool in the presence of different amounts of the test antibiotic (i.e. step (b) of the method of the invention). In some embodiments, the relative transcription initiation rate of Tn_{A}P1 is at least 3 times, at least 100 times, at least 1000 times or at least 10000 times higher than that of Tn_{A}P3 under these conditions.

Each promoter includes: (a) a Pribnow box (−10 element); (b) a −35 element and (c) an UP element. Those skilled in the art are able to readily identify promoters having the required relative strengths by sequence analysis and/or in vitro or in vivo assays using expression constructs.

For example, suitable promoters can be engineered or selected as described in Rhodius et al. (2011) Nucleic Acids Research: 1-18.

Moreover, the rapid application of next generation sequencing to RNA-seq is now providing a wealth of high-resolution information of transcript start sites at a genomic level, which greatly simplifies the identification of promoter sequences in any given Gram-negative bacterium. This permits the construction of descriptive promoter models for entire genomes. RNA-seq also provides quantitative information on transcript abundance and hence promoter strength, which enables the construction of promoter strength models that can then be used for predictive promoter strength rankings (see Rhodium et al. (2011) Nucleic Acids Research: 1-18).

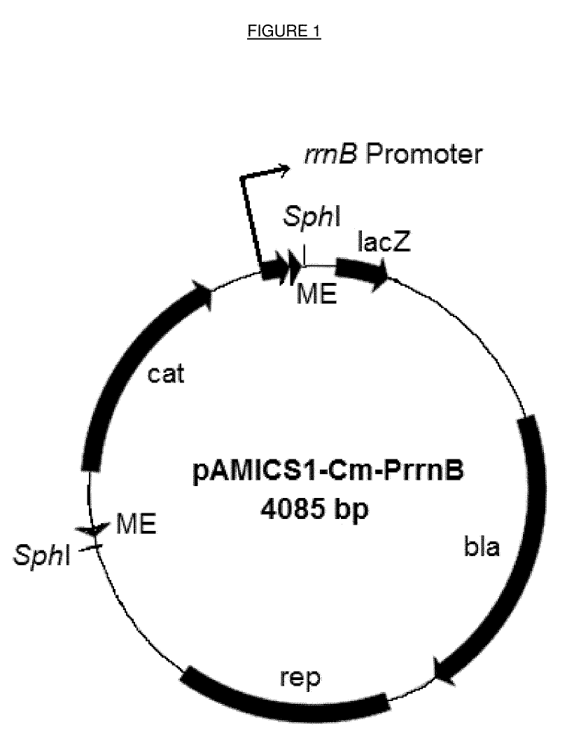

Suitable promoters can also be identified by assay. For example, a series of plasmids based on that shown in FIG. 1 can be used to test promoter strength empirically. FIG. 1 is a genetic map of plasmid pAMICS1-Cm-PrrnB. The transposon is flanked by the mosaic ends, ME, and the outward oriented rrnB promoter in indicated. Other components of the plasmid outside of the transposon are from the plasmid vector pBluescript (Agilent), lacZ, beta-galactosidase subunit; bla, beta-lactamase coding for ampicillin resistance; rep, pBR322 replication origin; cat, chloramphenicol acetyltransferase coding for chloramphenicol resistance.

Briefly, the promoter to be tested is placed upstream of an antibiotic resistance gene (in this case Kanamycin) and then transformed into the relevant bacteria. General cloning assembly and plasmid amplification can be carried out in E. coli (facilitated by the ampicillin resistance gene and the pBR322 ori) and the activity of the promoter in the target bacterium can then be assayed by generating a killing curve with Kanamycin—a very high level promoter gives more KmR expression and therefore survival at a higher antibiotic concentration. The plasmid series is designed to be modular so that the origin of replication, resistance gene(s) and promoter can be easily switched.

Suitable promoters include the E. coli rplJ (large ribosomal subunit protein; moderate strength promoter); tac (artificial lac/trp hybrid; strong promoter) and rrnB (ribosomal RNA gene promoter; very strong promoter) promoters.

As used herein, the terms P_{rpU} and P_{rrnB} specifically refer to the E. coli promoters for the 50S ribosomal subunit protein L10 and 16S ribosomal RNA genes, respectively. Orthologues of these (and other) E. coli promoters from other Gram-negative bacteria can also be used, including in particular the orthologous Pseudomonas aeruginosa or Acinetobacter baumannii promoters.

For example, the orthologous Acinetobacter baumannii gene corresponding to the E. coli rrnB P_{rrnB} has the gene symbol A1S_r12 and encodes the Acinetobacter baumannii 16S ribosomal RNA gene, so that the corresponding orthologous promoter is herein designated P_{(A1S}_{_}ᵣ₁₂₎. Thus, when the method is applied to Acinetobacter baumannii, Tn_{P1} may be P_{(A1S}_{_}ᵣ₁₂₎.

Similarly, when the method of the invention is applied to Pseudomonas aeruginosa, Tnp₂ may be the 16S ribosomal RNA gene promoter from P. aeruginosa (i.e. Ps.P_{rrnB}) while Tn_{P3} may be selected from the rpsJ (small (30S) ribosomal subunit S10 protein) gene promoter from P. aeruginosa (i.e. Ps.P_{rpsJ}) and the E. coli P_{rrnB}.

### RNA Transcript Profiling

Sequencing mRNA transcripts produced by Tn_{A}P in each of said test cultures permits an mRNA profile to be produced for each of the test cultures.

The transcript profile may comprise:
the sequences of said mRNA transcripts produced by TnAP; and/or
the start and finish of mRNA transcripts produced by TnAP; and/or
the lengths of said mRNA transcripts produced by TnAP; and/or
the relative abundance of said mRNA transcripts produced by TnAP; and/or
the site of transcription on the bacterial DNA; and/or
whether the mRNA transcripts produced by TnAP is sense or antisense with respect to the bacterial DNA; and/or
whether the mRNA transcripts produced by TnAP correspond to ORFs with respect to the bacterial DNA; and/or
whether the mRNA transcripts produced by TnAP encode bacterial proteins and/or protein domains.

In preferred embodiments, said mRNA transcript profile comprises a determination of the sequence of one or more antisense transcripts arising from TnA insertion into an insertion site within a noncoding, anti-sense strand of the DNA of said bacterium.

Any suitable high-throughput massively parallel sequencing technique can be used, including those based on sequencing-by-synthesis (SBS) and nanopores, and there are many commercially available sequencing platforms that are suitable for use in the methods of the invention. SBS-based sequencing platforms are particularly suitable for use in the methods of the invention: for example, the Illumina™ system is generates millions of relatively short sequence reads (54, 75 or 100 bp) and is particularly preferred.

Illumina™ sequencing, or other technologies, based on reversible dye-terminators may be used. Here, DNA molecules are first attached to primers on a slide and amplified so that local clonal colonies are formed (bridge amplification). Four types of ddNTPs are added, and non-incorporated nucleotides are washed away. Unlike pyrosequencing, the DNA can only be extended one nucleotide at a time. A camera takes images of the fluorescently labeled nucleotides then the dye along with the terminal 3′ blocker is chemically removed from the DNA, allowing a next cycle.

Other systems capable of short sequence reads include SOLiD™ and Ion Torrent technologies (both sold by Applied Biosystems™). SOLiD™ technology employs sequencing by ligation. Here, a pool of all possible oligonucleotides of a fixed length are labelled according to the sequenced position. Oligonucleotides are annealed and ligated; the preferential ligation by DNA ligase for matching sequences results in a signal informative of the nucleotide at that position. Before sequencing, the DNA is amplified by emulsion PCR. The resulting bead, each containing only copies of the same DNA molecule, are deposited on a glass slide. The result is sequences of quantities and lengths comparable to Illumina sequencing.

Ion Torrent Systems Inc. have developed a system based on using standard sequencing chemistry, but with a novel, semiconductor based detection system. This method of sequencing is based on the detection of hydrogen ions that are released during the polymerisation of DNA, as opposed to the optical methods used in other sequencing systems. A microwell containing a template DNA strand to be sequenced is flooded with a single type of nucleotide. If the introduced nucleotide is complementary to the leading template nucleotide it is incorporated into the growing complementary strand. This causes the release of a hydrogen ion that triggers a hypersensitive ion sensor, which indicates that a reaction has occurred. If homopolymer repeats are present in the template sequence multiple nucleotides will be incorporated in a single cycle. This leads to a corresponding number of released hydrogens and a proportionally higher electronic signal.

### Functional Assessment of Putative Essential Genes

The profiling of the mRNA transcripts may comprise a determination of whether the mRNA transcripts produced by Tn_{A}P encode bacterial proteins and/or protein domains.

This may comprise characterization of the mRNA transcripts by various techniques which directly or indirectly assess the function of the RNA per se (or the encoded proteins).

Suitable techniques include bioinformatics, where the (full or partial) sequence of the mRNA and/or its cognate amino acid sequence is used to interrogate sequence databases containing information from the bacterium assayed and/or other species in order to identify genes (e.g. orthologous genes in other species) for which biochemical function(s) have already been assigned.

Suitable bioinformatics programs are well known to those skilled in the art and include the Basic Local Alignment Search Tool (BLAST) program (Altschul et al. (1990) J. Mol. Biol. 215: 403-410 and Altschul et al. (1997) Nucl. Acids Res. 25: 3389-3402). Suitable databases include, for example, EMBL, GENBANK, TIGR, EBI, SWISS-PROT and trEMBL.

Alternatively, or in addition, the (full or partial) sequence of the mRNA and/or its cognate amino acid sequence is used to interrogate a sequence database containing information as to the identity of essential genes which has been previously constructed using the conventional Tn-seq methods described in the prior art (e.g. as described in Gawronski et al. (2009) PNAS 106: 16422-16427; Goodman et al. (2009) Cell Host Microbe 6: 279-289; van Opijnen et al. (2009) Nat. Methods 6: 767-772; Langridge et al. (2009) Genome Research 19: 2308-2316; Gallagher et al. (2011) mBio 2(1):e00315-10) and/or the techniques described in WO 01/07651 (the contents of which are hereby incorporated by reference).

Alternatively, or in addition, essentiality can be imputed by eliminating the possibility that a putative essential gene acts as an antibiotic resistance gene. For example, the (full or partial) sequence of the putative essential gene is used to interrogate sequence databases containing sequence information of genes previously identified as antibiotic resistance genes using the Tn-seq methods described in e.g. Gawronski et al. (2009) PNAS 106: 16422-16427; Goodman et al. (2009) Cell Host Microbe 6: 279-289; Langridge et al. (2009) Genome Research 19: 2308-2316 or Gallagher et al. (2011) mBio 2(1):e00315-10. Antibiotic resistance genes may be identified in such methods as a class of niche-specific/conditionally essential genes.

### EXEMPLIFICATION

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Preparation of Bacteria for Electroporation

Bacteria are grown in 2×TY broth to an OD₆₀₀ of 0.3-0.5. Cells are then harvested and washed three times in ½ original culture volume 10% glycerol and resuspended in 1/1000 original culture volume 10% glycerol and stored at −80° C.

### Preparation of Transposomes

Transposon DNA (a derivative of EZ-Tn5™<R6Kγori/KAN-2> possessing an internal lac promoter is amplified using 5′ phosphorylated oligonucleotides 5′-CTGTCTCTTATACACATCTCCCT and 5′-CTGTCTCTTATACACATCTCTTC with Q5 high fidelity DNA polymerase (New England Biolabs). As an alternative, the internal lac promoter can be replaced with a tac promoter (as described supra). Two hundred nanograms of this DNA is then incubated with EZ-Tn5™ transposase (Epicenter Biotechnologies) at 37° C. for 1 h then stored at −20° C. at a DNA concentration of 20 ng/μl.

### Generation of Mutant Bacterial Pools

Sixty microliters of cells (previously stored at −80° C. are mixed with 0.2 μl (4 ng) of transposomes and electrotransformed in a 2-mm electrode gap cuvette using a Bio-Rad GenePulser II set to 2.4 kV, 25 μf, and 200Ω. Cells are resuspended in 1 mL of SOC medium (Invitrogen) and incubated at 37° C. for 2 h then spread on L-agar bacteroiological growth medium supplemented with an appropriate concentration of kanamycin. The concentration of kanamycin used is strain dependent and determined empirically

After incubation overnight at 37° C., the number of colonies on several plates is estimated by counting a proportion of them, and from this the total number of colonies on all plates is estimated conservatively. Kanamycin resistant colonies are harvested by resuspension in sterilized deionized water using a bacteriological spreader. Resuspended cells from several electroporations are then pooled to create mutant library mixtures estimated to include over 1 million mutants.

### Identifying Antibiotic Target Gene(s)

Eight cultures of 10 ml broth medium are prepared, six of which are supplemented, in duplicate, with the test antibiotic at a concentrations 0.5, 1 and 2×MIC. Any required promoter inducer is also be added to the medium at this time to ensure active transcription directed into the chromosomal DNA from the transposon sequence.

Assuming a transposon mutant pool of 1 million mutants, 10⁸-10⁹ cfu of the pool are used to inoculate each culture. Cultures are grown to stationary phase and fresh cultures prepared and inoculated with 10⁸-10⁹ cfu from the first cultures. Cells are harvested from this secondary culture during exponential growth for RNA extraction and the remaining culture grown to stationary phase and cells harvested for extraction of genomic DNA.

RNA is sequenced, for example, using the Life technologies Proton platform. After total RNA was extracted using RNeasy mini-kit (Qiagen), RNA samples were 3′ tagged by ligation, of a 5′ phosphorylated, 3′ dideoxy terminated DNA oligonucleotide RP-1 (phosphorylation-5′-AAC CTG GAC ATA CGC ATC CG(ddA)-3′) to the 3′ end of the RNA using T4 RNA ligase (NEB). The 3′ tagged RNA molecules were then converted to cDNA, using a reverse complement of the RP-1 oligo, RP-1-rev (5′-CGG ATG CGT ATG TCC AGG TT-3′) by incubating with M-MLV reverse transcriptase RNaseH minus (Promega) at 30° C. for 30′, 45° C. for 30′ and 50° C. for 30′, then treated with 100 U/μl RNaseA and 0.02 U/μl RNaseH to degrade any surviving RNA. The cDNA was then amplified by PCR with phusion DNA polymerase (Thermo) using an oligo with sequence specificity to the transposon in a region common to all promoters, TN-5-fwd (5′-CGA CTC ACT ATA GGG AGA TGT-3′) and RP-1-rev as the reverse primer. PCR reactions were cleaned up using a Wizard SV reaction purification kit (Promega) and then sequencing performed on an Ion Torrent (Life Technologies) Proton sequencer, after fragmented library preparation. The sequencing runs were used to determine the position and abundance of the transposon inserts within the genome and visualised using Artemis genome browser (Sanger).

### Essential Genes/Genes Becoming Essential on Ceftriaxone Exposure

Libraries of transposon mutants in bacteria were generated as described above to produce at least 3 libraries with different strength outward facing promoters. These libraries were then pooled to ensure equal spread of the mutations and grown in the presence of ceftriaxone at 2× the MIC at 37° C. in appropriate growth media at a ratio that ensured 100's of copies of each transposon for 16 h.

The cells were then subcultured by 1:100 dilution into fresh media containing 2×MIC ceftriaxone and then grown into exponential phase (OD₆₀₀ of 0.4-0.5, usually taking 3-4 hours) at 37° C. At this point 2×0.5 ml of culture was harvested by centrifugation and both total RNA and genomic DNA extraction using RNeasy mini-kit (Qiagen) or Wizard SV genomic DNA kit (Promega).

DNA was quantified by UV spectrophotometry and 4.5 μg fragmented by sonication on a Covaris M220 and library prepared by end repair and ligation of specific amplification tags to the fragmented DNA, followed by PCR amplification for transposon sequences and the ligation tags. The libraries were then sequenced on Ion torrent proton sequencers. RNA samples were 3′ tagged with either a polyA tail using E. coli poly(A) polymerase (NEB) or by ligation, of a 5′ phosphorylated, 3′ dideoxy terminated DNA oligonucleotide RP-1 (phosphorylation-5′-AAC CTG GAC ATA CGC ATC CG(ddA)-3′), to the 3′ end of the RNAusing T4 RNA ligase (NEB). All oligonucleotide used were ordered from Integrated DNA technologies with HPLC purification. The 3′ tagged RNA molecules were then converted to cDNA, using a poly dT₂₀ oligo for E. coli poly(A) polymerase tagged RNA, or alternatively reverse complement of the RP-1 oligo, RP-1-rev (5′-CGG ATG CGT ATG TCC AGG TT-3′) by incubating with M-MLV reverse transcriptase RNaseH minus (Promega) at 30° C. for 30′, 45° C. for 30′ and 50° C. for 30′, then treated with 100 U/μl RNaseA and 0.02 U/μl RNaseH to degrade any surviving RNA. The cDNA was then amplified by PCR with phusion DNA polymerase (Thermo) using an oligo with sequence specificity to the transposon in a region common to all promoters, TN-5-fwd (5′-CGA CTC ACT ATA GGG AGA TGT-3′) and either RP-1-rev or poly dT₁₂ as the reverse primer. PCR reactions were cleaned up using a Wizard SV reaction purification kit (Promega) and then library preparation in the same manner as the genomic DNA. The sequencing runs were used to determine the position and abundance of the transposon inserts within the genome and visualised using Artemis genome browser (Sanger).

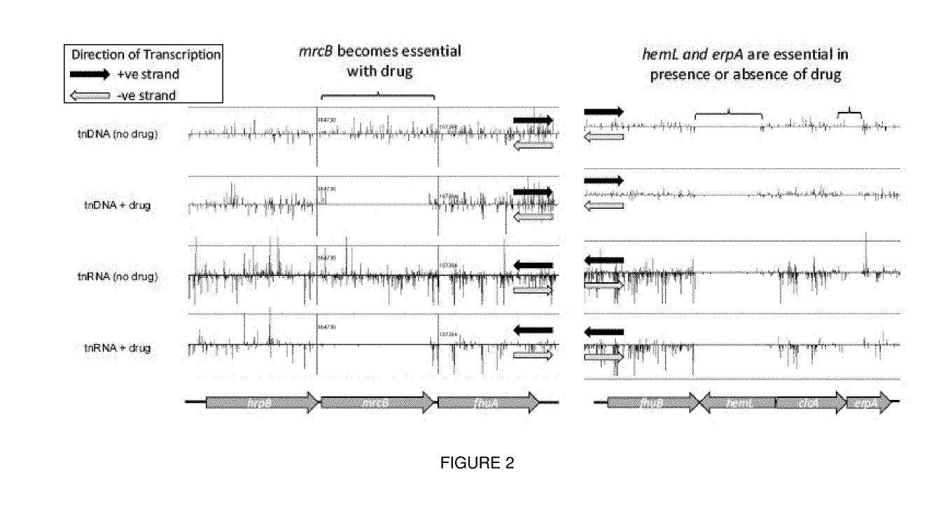

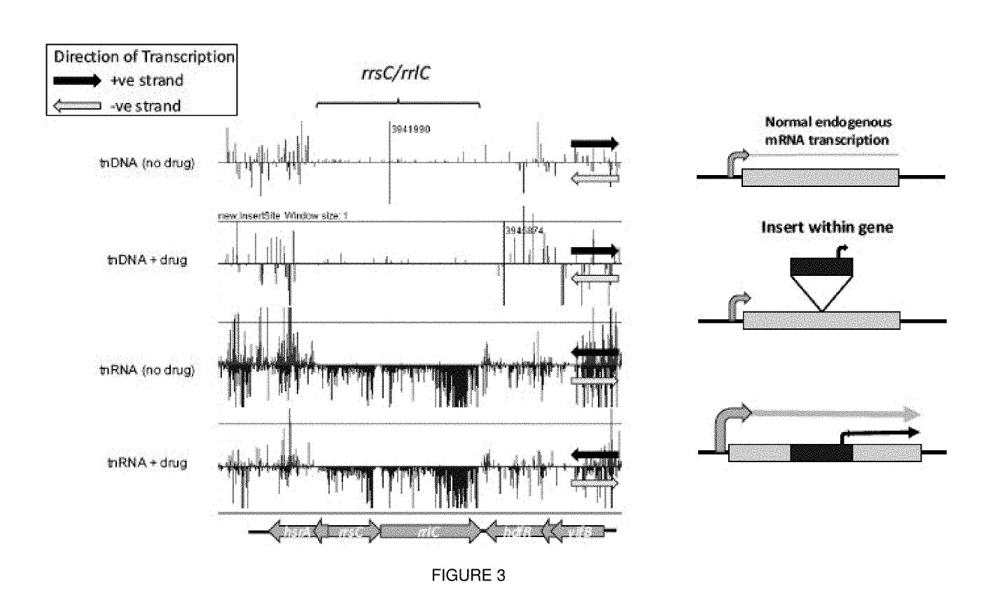

As shown in FIG. 2, the RNA sequencing provides similar positional data to the DNA sequencing for the location of transposons and in many cases few inserts are seen within genes essential for growth or that become essential with drug treatment. Additionally it also shows levels of transcription of genomic regions. For example were genomic inserts are rare there are still abundant RNA inserts detected suggesting very high transcription in this region, either the gene is always heavily transcribed, or the response to the insertion is massive increases in expression to try and compensate for the reduced/loss of functionality of the disrupted gene, as well as some transcription from the transposon itself (FIG. 3).

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A method for characterizing the effect of an antibiotic on a bacterium, the method comprising the steps of:
(a) generating a pool of mutant bacteria by transposon mutagenesis of a culture of said bacterium with an activating transposon (Tn_{A}) which comprises an outward-facing promoter (Tn_{A}P) capable of increasing transcription of a gene at or near its insertion site in the DNA of said bacterium;
(b) growing bacteria from the mutant pool in the presence of different amounts of said antibiotic to produce two or more test cultures;
(c) sequencing mRNA transcripts produced by Tn_{A}P in each of said test cultures to produce an mRNA transcript profile for each of the test cultures; and
(d) comparing the mRNA transcript profiles of the test cultures.

2. The method of claim 1 wherein said mRNA transcript profile comprises a determination of:
(a) the sequences of said mRNA transcripts produced by Tn_{A}P; and/or
(b) the start and finish of mRNA transcripts produced by TnAP; and/or
(c) the lengths of said mRNA transcripts produced by Tn_{A}P; and/or
(d) the relative abundance of said mRNA transcripts produced by Tn_{A}P; and/or
(e) the site of transcription on the bacterial DNA; and/or
(f) whether the mRNA transcripts produced by Tn_{A}P is sense or antisense with respect to the bacterial DNA; and/or
(g) whether the mRNA transcripts produced by Tn_{A}P correspond to ORFs with respect to the bacterial DNA; and/or
(h) whether the mRNA transcripts produced by Tn_{A}P encode bacterial proteins and/or protein domains; and/or
(i) sequence of one or more antisense transcripts arising from Tn_{A} insertion into an insertion site within a noncoding, anti-sense strand of the DNA of said bacterium.

3. (canceled)

4. The method of claim 1 further comprising the step of isolating mRNA from said test cultures of step (b).

5. The method of claim 1 wherein in step (c) the sequences of said mRNA transcripts produced by Tn_{A}P are determined by a method comprising the steps of:
(a) isolating RNA from said test cultures of step (b);
(b) ligating a 3′ terminal linear nucleic acid linker of predetermined sequence to said isolated RNA;
(c) synthesising cDNA primed by the reverse complement of said nucleic acid linker to produce a cDNA-RNA hybrid;
(d) degrading the RNA strand of the cDNA-RNA hybrid, and optionally any remaining total RNA, to produce ss cDNA;
(e) amplifying the cDNA using a TnA-specific primer and a the reverse complement of step (c); and
(f) sequencing the amplified cDNA of step (f).

6. The method of claim 1 wherein in step (c) the sequences of said mRNA transcripts produced by Tn_{A}P are determined by a method comprising the steps of:
(a) isolating mRNA from said test cultures of step (b);
(b) ligating a terminal linear nucleic acid linker to said isolated mRNA and covalently closing the molecule;
(c) synthesising cDNA primed by said nucleic acid linker to produce a cDNA-mRNA hybrid;
(d) degrading the RNA strand of the cDNA-RNA hybrid to produce ssDNA;
(e) synthesising the complementary DNA strand of the linear or circular ssDNA using a Tn_{A}-specific primer to produce duplex DNA;
(f) amplifying the DNA using the Tn_{A}-specific primer and a linear specific linker; and
(g) sequencing the amplified DNA of step (f), optionally by a method comprising sequencing-by-synthesis (SBS) biochemistry.

7. The method of claim 1 wherein the pool of mutant bacteria comprises: (a) at least 0.5×10⁵ mutants; (b) at least 5×10⁵ mutants; (c) at least 1×10⁶ mutants; (d) 0.5×10⁶ to 2×10⁶ mutants; or (e) about 1×10⁶ mutants.

8. The method of claim 1 wherein the transposon mutagenesis step (a) yields an insertion rate of at least one transposon per 50 base pairs of bacterial DNA.

9. The method of claim 1 wherein the transposon mutagenesis step (a) yields an insertion rate of at least one transposon per 25 base pairs of bacterial DNA.

10. The method of claim 1 wherein the transposon mutagenesis step (a) yields an insertion rate of at least one transposon per 15 base pairs of bacterial DNA.

11. The method of claim 1 wherein the transposon mutagenesis step (a) yields an insertion rate of at least one transposon per 10 base pairs of bacterial DNA.

12. The method of claim 1 wherein the insertion site in the DNA of said bacterium is within the chromosomal (genomic) DNA thereof or extra-chromosomal DNA thereof.

13. (canceled)

14. The method claim 1 wherein the transposon mutagenesis of step (a) occurs in vivo.

15. The method of claim 1 wherein the transposon mutagenesis of step (a) occurs in vitro.

16. The method of claim 1 wherein the bacterium is a Gram-positive bacterium Gram-negative bacterium or a bacterium of which the gram reaction is indeterminate.

17-20. (canceled)

21. The method of claim 1 wherein bacteria are grown from the mutant pool in step (b) by inoculating growth medium with 10⁷ to 10⁹ cfu from the mutant pool.

22. The method of claim 1 wherein bacteria are grown from the mutant pool in step (b) in the presence of antibiotic at a concentration of about 0.5, about 1 and about 2×MIC to produce at least three test cultures.

23. The method of claim 1 for identifying an essential gene which serves as an antibiotic target in said bacterium, wherein in step (d) the mRNA transcript profiles of the test cultures are used to identify a putative essential gene serving as a target of said antibiotic in said bacterium.

24. The method of claim 1 wherein in step (a) said pool of mutant bacteria is generated by transposon mutagenesis with a plurality of Tn_{A}s comprising: (i) a Tn_{A} comprising an outward-facing first promoter Tn_{A}P1; (ii) a Tn_{A} comprising an outward-facing second promoter Tn_{A}P2; and (iii) a Tn_{A} comprising an outward-facing third promoter Tn_{A}P3, wherein the relative strength of said promoters is: Tn_{A}P1>Tn_{A}P2>Tn_{A}P3; such that transposon insertion into bacterial DNA generates a pool of mutant bacteria in which one or more genes are transcribed from Tn_{A}P1, one or more genes are transcribed from Tn_{A}P2 and one or more genes are transcribed from Tn_{A}P3.

25. A method of identifying an antibiotic comprising identifying an essential gene which serves as a target of said antibiotic according to a method as defined in claim 1.

26. A process for producing an antibiotic comprising the method as defined in claim 25.

27-28. (canceled)


SEQUENCE LISTING (5 sequences)

SEQ ID NO: 1 - DNA, 23 bp, Artificial Sequence
  Synthetic Polynucleotide
ctgtctcttatacacatctccct

SEQ ID NO: 2 - DNA, 23 bp, Artificial Sequence
  Synthetic Polynucleotide
ctgtctcttatacacatctcttc

SEQ ID NO: 3 - DNA, 21 bp, Artificial Sequence
  Synthetic Polynucleotide
aacctggacatacgcatccga

SEQ ID NO: 4 - DNA, 20 bp, Artificial Sequence
  Synthetic Polypolynucleotide
cggatgcgtatgtccaggtt

SEQ ID NO: 5 - DNA, 21 bp, Artificial Sequence
  Synthetic Polynucleotide
cgactcactatagggagatgt